(19) United States
(12) Patent Application Publication
(10) Pub. No.: US 2003/0180387 A1
(43) Pub. Date: Sep. 25, 2003

(54) Method for increasing the antioxidative potential of selenium-containing aqueous solutions

(76) Inventors: Peter Kossler, Mariapfarr (AT); Norbert Fuchs, Mariapfarr (AT); Bodo Kuklinski, Rostock (DE); Raimund Schiefer, Tamsweg (AT)

Correspondence Address: FULBRIGHT & JAWORSKI L.L.P., 600 CONGRESS AVE., SUITE 2400, AUSTIN, TX, 78701, US

(21) Appl. No.: 10/297,533

(22) PCT Filed: May 23, 2001
(86) PCT No.: PCT/AT01/00162

(30) Foreign Application Priority Data
Jun. 5, 2000 (AT) A 978/2000

(51) Int. Cl. A61K 33/04; A61K 31/19

(52) U.S. Cl. 424/702; 514/557; 514/574

(57) ABSTRACT

There is disclosed a method for increasing the antioxidant potential of selenium-containing aqueous solutions, wherein a selenium-containing aqueous solution is supplemented with pharmaceutically acceptable or food-compatible acids, selenium-containing preparations as well as the use of such preparations.

The invention relates to a method for increasing the antioxidant potential of selenium-containing aqueous solutions as well as pharmaceutically administrable or food-compatible selenium preparations.

All metabolic processes in organic living beings (plants, animals, humans) in the sense of growth, differentiation and energy processes constitute interplays between reductive and oxidative processes on the biochemical level. After all, these “redox procedures” are expressions of the electron transfer of biochemical reduction equivalents such as, e.g., NADH+H⁺(electron donor) to atmospheric molecular oxygen as an oxidizing agent (electron acceptor). The oxidation of our nutrients (fats, carbohydrates, proteins, oxygen) serves the permanent support and evolution of our biological structures.

On the other hand, it is exactly our cellular and subcellular structures, the tissues and organs formed thereof and, last but not least, each organic individuum which, in their entirety, are comprised of those structures (nutrients) which have to be constantly supplied externally for the support and evolution of living organisms, oxidized for the recovery of energy, but which, at the same time, also must serve to support the functional, anatomical and histological structures. Thus, these biological structures are actually as oxidable as those nutrients which have to be oxidized to conserve our vital energy. In order to inhibit the “autooxidation” of biological structures, the organic living organism uses endogenic and exogenic “antioxidants”. Endogenic antioxidants include, inter alia, enzymes and enzyme systems such as superoxide dismutase, catalases, peroxydases, cholesterol and reduced glutathion, while exogenic antioxidants comprise, for instance, vitamin A, β-carotene, vitamin E, vitamin C or selenium.

The measure of the “antioxidant capacity”, i.e., the readiness to transfer electrons to other atoms and molecules, is quantitatively expressed by what is called “reduction potential” .(standard redox potential). The following Table gives a survey on the standard redox potentials of some endogenic and exogenic antioxidants of organic living beings:

Standard redox potentials of some nutrients

| Eₒ (Volt) | System |
|---|---|
| +0.82 | O₂/H₂O |
| +0.366 (basic environment) | selenite |
| +0.300 | tocopherol (vitamin E) |
| +0.100 | ubiquinone (coenzyme Q₁₀) |
| +0.08 | ascorbic acid |
| +0 (+0.16 to −0.02 V) | flavonoids |
| −0.12 | riboflavin (vitamin B₂) |
| −0.22 | cystin/cystein |
| −0.23 | G SH/GSSG |
| −0.29 | thioctic acid (α-liponic acid) |
| −0.32 | NADH + H⁺/NAD |
| −0.740 (acidic environment) | selenite |

Antioxidants are, thus, atoms and molecules (for the human organism, primarily nutrient molecules and enzyme complexes) which react with metabolic radicals more rapidly than biological structures. Thus, they protect our cell, gene and connective tissue structures by trapping metabolic trigger sparks (radicals, peroxides) before the latter attack, for instance, unsaturated fatty acids of our biomembranes or sulphur-containing components of vital structural or enzymatic proteins. As is apparent from the above Table, certain elements such as, e.g., selenium change their standard redox potentials by changing the pH environment in which these compounds are dissolved.

Selenium is an essential micronutrient for higher animals and man. It has a protective function for proteins against oxidation caused, for instance, by glutathion peroxidase, which is contained in the active center of the aminoacid selenocystein. A selenium deficiency is associated with rheumatism and grey cataract, and the Keshan disease, which is common in some areas of China, is supposed to go back to selenium deficiency symptoms. Selenites are able to increase the effect of vitamin E and induce mercury and cadmium detoxication. A protective effect of selenium against carcinogens has also been reported.

On the other hand, selenium in higher concentrations has toxic effects, its toxicity supposedly going back to its ability to displace the sulphur contained in proteins. Excretion, as a rule, occurs via the kidney and the intestine in the form of selenates. Disorders of the human body will be caused if the daily nutrition contains more than 1 μg selenium/g (a minimum content of 0.02 μg selenium/g being required to prevent deficiency symptoms). Overall, the human body contains approximately 10 to 15 μg selenium.

Toxication will occur also in animals if animal nourishment contains more than 5 to 10 μg selenium/g, involving, for instance, growth inhibition, the loss of hair, the softening of horns and hoofs, and the loss of feathers with birds. Yet, selenium is necessary also for animals, when raising chickens, turkey hens and pigs, as well as to avoid specific diseases of domestic cattle, in particular sheep. Sodium selenite and sodium selenate are, therefore, required as supplements for mixed provender or for fertilizers of pastures, since the natural selenium contents of animal and vegetable feed are frequently insufficient, or the element is released in an insufficent manner.

It is the object of the present invention to improve selenium-containing preparations and use them in a food/feed-technological context as well as a pharmaceutical context, and to enhance their activities in these fields.

In accordance with the invention, this object is achieved by a method for increasing the antioxidant potential of selenium-containing aqueous solutions, which is characterized in that a selenium-containing aqueous solution is supplemented with pharmaceutically acceptable or food-compatible acids.

It has been shown that a decrease of the pH of selenium-containing solutions, in particular sodium selenite and selenate solutions, exhibit a strongly increased antioxidant potential as compared to non-pH-reduced solutions. The solutions prepared according to the invention also show surprising therapeutic effects, primarily with diseases induced by radicals and peroxides.

Preferably, a selenite or selenate solution is, therefore, used as said selenium-containing aqueous solution.

Acidifying agents for the selenium-containing solution comprise, in particular, acidifying agents that are generally recognized as safe to both man and animal, such as, e.g., citric acid, acetic acid, malic acid, carbonic acid, various fruit acids and mixtures thereof.

The present invention also relates to a preparation comprising

a pharmaceutically administrable or food-compatible form of selenium, namely selenite or selenate, and

a pharmaceutically acceptable or food-compatible acid selected from citric acid, acetic acid, malic acid, carbonic acid, various fruit acids and mixtures thereof.

The preparation according to the invention has an enhanced antioxidant potential, thus exhibiting positive properties for both man and animal when applied in a food-technological and feed-technological context. In connection with the pharmaceutical use of selenium compounds, also the pharmaceutical action is enhanced by the preparation according to the invention, or even new pharmaceutical applications for selenium compounds are opened up.

In a preferred manner, acidification of the selenium-containing agent provides a pH of below 7.0, preferably below 5.0, in particular below 4.0. Agents according to the invention which are particularly preferred have a pH ranging from 6.0 to 2.0, in particular 3.0 to 2.5.

In the context of the preparation according to the invention, selenium is made available in the form of selenite or in the form of selenate. For different purposes of use, the dimethylselenide, selenomethionin, selenocystein forms or mixtures of these forms are suitable as well.

The preparation according to the invention may be provided not only in an aqueous solution. Preferred other forms comprise gels or emulsions, which have proved to be excellently suitable, in particular, for pharmaceutical applications, enabling local topical application.

It goes without saying that the preparation according to the invention may additionally contain auxiliary substances like buffering agents, dyes, stabilizing agents or carrier substances and/or further active components such as, e.g., antibiotics, antiviral agents, antimyotics, analgetics or anti-inflammatory agents, said auxiliary substances being also usable in any desired combinations. The respective type of auxiliary substance and/or additional active component is a function of the respective use in each individual case.

The preparation according to the invention is particularly apt for pharmaceutical uses. Yet, also its use as a foodstuff or food supplement or as a feedstuff or feed supplement is preferred.

During the pharmaceutical application of the preparation according to the invention, it was surprisingly found that it is, above all, effective in the prevention or treatment of peroxidic and free-radical diseases.

In a preferred manner, the preparation according to the invention is, therefore, used to prepare a drug designed to prevent or treat viral diseases, preferably herpes infections, in particular herpes simplex infections. On the other hand, the preparation according to the invention also has been shown to be extremely active in the prevention or treatment of pigmental moles (caused by lipofuszin depositions).

The invention will now be explained in more detail by way of the following examples to which it is, however, not limited.

### EXAMPLE 1

### Preparation of an Acidified Sodium Selenite Solution

An acidified sodium selenite solution having the following composition (per 100 ml) was prepared:

| | |
|---|---|
| sodium selenite pentahydrate | 0.111 g |
| maltodextrin | 0.5 g |
| citrus aroma | 0.1 g |
| citric acid | 0.5 g |
| food dye | 0.01 g |
| potassium sorbate | 0.1 g |
| sodium benzoate | 0.05 g |
| aqua destillata | 99.29 g |

### EXAMPLE 2

### Treatment of Herpes Simplex Infections

Twelve adult patients (seven female and five male patients) as well as eight childen (four females and four males) diagnosed to suffer from stomatitis herpetica/aphtosa were treated buccally with five drops five times a day (childen receiving the solution at a ten-fold dilution) and, at the same time, externally (the affected sites being dabbed with the droplets five times a day), usually over a period of seven days. Seven out of eight children, in addition to the antioxidant selenium therapy, were prescribed local anesthetics and/or antibiotics and/or antimycotics and/or pain-relieving and anti-inflammatory drugs, but no additional antiviral therapy.

Nine out of the twelve adults were treated exclusively with the strongly antioxidant selenium drops, one of the twelve patients receiving an antiviral drug (aciclovir) in addition to the drops. The results are illustrated in the Table below.

| | | | | | |
|---|---|---|---|---|---|
| Patient | | | previous | side- | Treatment |
| No. | Sex | Incidence | treatment | effects | results |

Herpes labialis therapy

| | | | | | |
|---|---|---|---|---|---|
| 1 | m | 1 y | local | none | 1 |
| | | | ointment | | |
| | | | therapy | | |
| 2 | m | mth. | aciclovir | none | 1 |
| 3 | m | 3-5 y | aciclovir | none | 1 |
| 4 | f | 3 y | aciclovir | none | 1 |
| 5 | m | — | — | — | 1 |
| 6 | f | 3 y | famvir | none | 2 |
| 7 | f | mth. | aciclovir | none | 1 |
| 8 | f | 3-5 y | aciclovir | none | 1 |
| 9 | f | 3 y | aciclovir | none | 1 |
| 10 | f | 3 y | aciclovir | none | 1 |

Stomatis herpetica/aphtosa therapy

| | | | | | |
|---|---|---|---|---|---|
| 11 | m | — | xylocain | none | 1 |
| 12 | f | — | — | none | 2 |
| 13 | f | — | — | none | 1 |
| 14 | m | — | — | none | 1 |
| 15 | m | — | — | none | 1 |
| 16 | f | — | — | none | 2 |
| 17 | m | — | — | none | 1 |
| 18 | f | — | — | none | 2 |
| 19 | f | — | — | none | 2 |
| 20 | m | mth. | — | none | 2 |

m = male
f = female
mth. = monthly
1 y = 1 × per year
2 y = 2 × per year
3 y = 3 × per year
3-5 y = 3 to 5 × per year
1 = very good
2 = good
3 = insufficient

The examination reports of the examining physician showed excellent therapeutic results (itching disappeared and vesicles healed) already after three to seven days in nineteen out of twenty cases. Those male and female patients who suffered from herpes relapses, after the buccal and external application of the drops showed markedly improved relapse rates (extended intervals without relapses), or the complete disappearance of relapses, as compared to an aciclovir therapy.

### EXAMPLE 3

### Treatment of Pigment Moles

Pigment moles (socalled age spots) are due to an elevated deposition of radically and peroxidically destroyed protein, fatty acid and membrane fat structures in the subcutaneous tissue, appearing as locally delimited light- to dark-brown discolorations of apprximately pin size. Three adult persons (two female and one male persons) applied the selenium droplets described (by ribbing in five to ten drops five times a day on the affected sites on the back of the hand) over a period of two months. The application led to a marked reduction of the number of pigment moles and to a brightening of dark pigment moles, respectively.

1. A method for increasing the antioxidant potential of selenium-containing aqueous solutions, characterized in that a selenium-containing aqueous solution is supplemented with pharmaceutically acceptable or food-compatible acids.

2. A method according to claim 1, characterized in that a selenite or selenate solution is used as said selenium-containing aqueous solution.

3. A method according to claim 1 or 2, characterized in that the selenium-containing aqueous solution is supplemented with citric acid, acetic acid, malic acid, carbonic acid, various fruit acids or mixtures thereof.

4. A preparation comprising
a pharmaceutically administrable or food-compatible form of selenium, namely selenite or selenate, and
a pharmaceutically acceptable or food-compatible acid selected from citric acid, acetic acid, malic acid, carbonic acid, various fruit acids and mixtures thereof.

5. A preparation according to claim 4, characterized in that it is present in an aqueous solution and has a pH of below 7.0, preferably below 5.0, in particular between 3.0 and 2.5.

6. A preparation according to any one of claims 4 to 5, characterized in that it is present in the form of a gel or emulsion.

7. A preparation according to any one of claims 4 to 6, characterized in that it additionally contains auxiliary substances and/or further active components.

8. A preparation according to any one of claims 4 to 7, characterized in that it contains as said auxiliary substances buffering agents, dyes, stabilizing agents, carrier substances or combinations thereof.

9. A preparation according to any one of claims 4 to 8, characterized in that it contains as said further active components antibiotics, antiviral agents, antimyotics, analgetics, anti-inflammatory agents or combinations thereof.

10. A preparation according to any one of claims 4 to 9 for pharmaceutical use.

11. The use of a preparation according to any one of claims 4 to 9 as a foodstuff or a food supplement.

12. The use of a preparation comprising a selenium-containing aqueous solution supplemented with pharmaceutically acceptable or food-compatible acids for the production of a drug designed to prevent or treat peroxidic diseases.

13. The use of a preparation comprising a selenium-containing aqueous solution supplemented with pharmaceutically acceptable or food-compatible acids for the production of a drug designed to prevent or treat free-radical diseases.

14. The use of a preparation comprising a selenium-containing aqueous solution supplemented with pharmaceutically acceptable or food-compatible acids for the production of a drug designed to prevent or treat viral diseases, preferably herpes infections, in particular herpes simplex infections.

15. The use of a preparation comprising a selenium-containing aqueous solution supplemented with pharmaceutically acceptable or food-compatible acids for the production of a drug designed to prevent or treat pigmental moles.
